(19) United States
(12) Patent Application Publication
(10) Pub. No.: US 2024/0368047 A1
(43) Pub. Date: Nov. 7, 2024

(54) MICROBIAL FERTILIZERS AND/OR ADDITIVES FOR NITROGEN-BASED FERTILIZERS

(71) Applicant: LOCUS SOLUTIONS IPCO, LLC, SOLON, OH (US)

(72) Inventors: SEAN FARMER, FT. LAUDERDALE, FL (US); YAJIE CHEN, SOLON, OH (US); MAJA MILOVANOVIC, NORTH ROYALTON, OH (US); CHASE MCANERNEY, SOLON, OH (US)

(21) Appl. No.: 18/248,379

(22) PCT Filed: Jan. 9, 2023
(86) PCT No.: PCT/US2023/060314

§ 371 Date: Apr. 8, 2023

Related U.S. Application Data
(60) Provisional application No. 63/297,767, filed on Jan. 9, 2022.
(60) Provisional application No. 63/314,664, filed on Feb. 28, 2022.

(51) Int. Cl. C05F 11/08; C05G 3/50; C12N 1/16; C12N 1/20; C12R 1/125; C12R 1/645

(52) U.S. Cl. CPC C05F 11/08; C05G 3/50; C12N 1/165; C12N 1/205; C12R 2001/125; C12R 2001/645

(57) ABSTRACT

The subject invention provides microbial fertilizer compositions for improving nitrogen uptake in plants and for reducing fertilizer usage in crop production.

### CROSS-REFERENCE TO RELATED APPLICATIONS

This application claims priority to U.S. Provisional Patent Application Ser. Nos. 63/297,767, filed Jan. 9, 2022, and 63/314,664, filed Feb. 28, 2022, each of which are incorporated herein by reference in their entireties.

### BACKGROUND OF THE INVENTION

Nitrogen is one of the most abundant elements on earth, and one of the most essential elements for supporting plant growth; however, a large portion of earth's nitrogen is found in the form of atmospheric nitrogen (N₂), which is not sufficiently usable by plants to fulfill their growth needs. Di-nitrogen is fairly inert due to the strength of a triple bond between its two nitrogen molecules. To break apart the molecule requires breaking each of these three bonds. Thus, plants require nitrogen in the form of organic nitrate or ammonium, which can be found in soil.

Often, however, soils do not contain sufficient amounts of these organic nitrogen compounds. Thus, fertilizers are essential for enhancing soil nutrient content and supporting the growth of crops. In addition to nitrogen, the basic nutrients that plants need for healthy growth include phosphorus (taken up as H₂PO₄⁻), and potassium (taken up as K⁺). Large amounts of nitrogen, phosphorus, and potassium nutrients are supplied mainly in the form of mineral fertilizers, either processed natural minerals or manufactured chemicals.

Despite the importance of mineral fertilizers for growing crops and producing food, over-use and/or long-term use of these compounds can cause harm to the health of consumers, animals and the environment. For example, nitrogen fertilizers can acidify soils, which can affect the growth of plants and other soil organisms that are not adapted to acidic environments. Extensive use of chemical nitrogen fertilizers may also inhibit the activity of natural nitrogen-fixing microorganisms, thereby decreasing the natural fertility of soils.

Mineral fertilizers may also introduce toxic substances into soil and produce. For example, phosphate fertilizers processed from rock phosphate often contain small amounts of toxic elements, such as cadmium, which may build up in soil and be taken up by plants.

The long-term use of mineral fertilizers may also cause environmental pollution. For example, soil erosion and leaching can cause nitrogen and phosphate fertilizers to contaminate ground water, which can run-off into rivers, lakes and oceans, leading to eutrophication.

Some attempts to reduce and/or eliminate the use of these mineral fertilizers have included utilizing organic fertilizers, which can be obtained from multiple different sources. Organic fertilizers can include, for example, farm wastes, such as crop residue or animal manure; animal parts, such as bone meal or blood; wood shavings and/or sawdust; and food or other home wastes, for example, in the form of compost. Nonetheless, organic fertilizers do not typically contain sufficient amounts and/or varieties of nutrients, and thus are not as effective in supporting plant growth without applying either large amounts of the organic fertilizers to soil or supplementing with mineral fertilizers.

Furthermore, organic fertilizers can pose their own environmental problems. For example, some organic fertilizers, if unprocessed, can contain pathogenic microorganisms, such as E. coli, Salmonella, and Coccidae. Organic fertilizers may also produce undesirable odors and can also contribute to the contamination and eutrophication of the natural water system. Accordingly, the problems with mineral fertilizers cannot be satisfactorily solved by substituting mineral fertilizers with organic fertilizers.

In addition to organic fertilizers, fertilizers utilizing microorganisms have been proposed as alternatives to mineral fertilizers. Certain microorganisms are capable of producing nitrogenases, which are enzymes and/or enzyme complexes that catalyze the reduction of N₂ to ammonia. Currently, nitrogenases are the only family of enzymes known to catalyze this reaction, which is a key step in the process of nitrogen fixation in soil.

In general, a complex comprising nitrogenase and a reductase enzyme carries out the process. The reductase portion provides electrons with reducing power, and the nitrogenase uses these electrons to reduce N₂. The transfer of electrons from the reductase to the nitrogenase component is coupled to the hydrolysis of adenosinetriphosphate (ATP) by the reductase.

Naturally-occurring nitrogen-fixing microorganisms including bacteria, such as Rhizobium, Azotobacter, and Azospirillum; and fungi, such as Aspergillus flavus-oryzae, have been utilized in biological fertilizers. Naturally-occurring microorganisms capable of solubilizing rock phosphate ore or other insoluble phosphates into soluble phosphates have also been utilized in microbial fertilizers, either separately or in combination with nitrogen-fixing microorganisms. Genetically modified bacterial strains have also been developed and utilized in microbial fertilizers to, for example, create more effective nitrogen fixing, phosphorus decomposing, and potassium decomposing bacterial strains for use in microbial fertilizers.

Like organic fertilizers, however, microbial fertilizers that are based on currently-available microorganisms are generally not sufficiently efficient to effectively replace mineral fertilizers. It is therefore important to develop new fertilizer compositions that can replace mineral fertilizers in supplying nutrients, particularly nitrogen, to crops.

### BRIEF SUMMARY OF THE INVENTION

The present invention provides a microbial plant fertilizer, which can be used for enhancing health, growth and/or yields of plants while replacing and/or reducing mineral fertilizer usage.

In one embodiment, the microbial fertilizer composition comprises a yeast, a bacterium, and/or a growth by-product thereof, and optionally, a fermentation medium in which the yeast or bacterium was cultivated. Preferably, the yeast or bacterium is capable of fixing atmospheric nitrogen and/or solubilizing nitrogen compounds.

In one embodiment, the yeast or bacterium according to the subject invention was not previously known to possess the ability to convert atmospheric nitrogen to nitrogenous compounds, such as nitrate, ammonia and/or ammonium, which can be used by plants as nutrients. In certain embodiments, the microbe's genome comprises a gene or a cluster of genes coding for the production of a nitrogen-fixing enzyme.

In certain embodiments, the nitrogen-fixing enzyme is a nitrogenase or an analog of a nitrogenase. In certain embodiments, the nitrogen fixing enzyme is a flavodoxin or an analog of a flavodoxin.

In certain embodiments, the term “nitrogen-fixing enzyme” includes enzyme complexes. For example, in certain embodiments, the microbe produces a nitrogen-fixing enzyme complex comprising a reductase enzyme in addition to a nitrogenase or flavodoxin, or analog thereof, which facilitates the transfer of electrons to the nitrogenase or flavodoxin, or analog thereof, for reduction of di-nitrogen.

In some embodiments, the composition can further comprise adenosinetriphosphate (ATP) or another electron donor for facilitating the nitrogen fixation reaction.

In certain preferred embodiments, the yeast is a Meyerozyma sp. yeast, for example, M. guilliermondii or M. caribbica (e.g., M. caribbica subsp. Locus). In a specific embodiment, the yeast is Meyerozyma sp. MEC14XN.

Advantageously, in certain embodiments, Meyerozyma sp. MEC14XN is endophytic and can provide nitrogen directly into plant roots; it can solubilize nitrogen; it is psycrophilic, capable of growing and reproducing in temperatures less than 10° C., more preferably less than 5° C.; it is salt-tolerant, capable of growing in high salinity soils; it is faster growing in soil and easier to obtain than traditional mycorrhizal fungi (which are mostly produced outside of the United States); it is capable of inhibiting plant-pathogenic Fusarium spp. fungi; and it is compatible with other plant-beneficial species of killer yeasts, such as Wickerhamomyces anomalus.

In certain preferred embodiments, the bacterium is a Bacillus sp. bacterium, for example, B. subtilis B4 NRRL B-68031, or B. amyloliquefaciens NRRL B-67928. In addition to nitrogen-fixation, these microbes are capable of producing lipopeptide biosurfactants and of surviving in conditions of high heat, low pH and low moisture.

In certain embodiments, the microbes of the subject invention are more effective at fixing and/or solubilizing nitrogen than known nitrogen-fixing microorganisms, such as, e.g., Azotobacter and commercially-available products such as Pivot Proven®. In certain embodiments, the microbes of the subject invention can be produced in greater quantities with greater ease than these known microbial products.

In one embodiment, the composition further comprises one or more sources of nutrients for the microorganism and/or for plants, such as an organic substrate component and/or an inorganic substrate component. The organic substrate component can comprise a carbon source or other nutrients for the microbial cells in the composition, for example, vegetable oil, glycerol, glucose, molasses, kelp extract, humic acid, fulvic acid, bone meal, blood meal, compost, corn gluten, potash, and/or manure from a variety of animals including horses, cows, pigs, chickens, and/or sheep. The inorganic substrate component can provide the microbial cells with minerals, materials and compounds containing, for example, nitrogen, phosphorus and/or potassium. The organic and inorganic substrate components may also provide the plants with other minerals such as, but not limited to, calcium magnesium and sulfur; and micronutrients, such as but not limited to, boron, copper, iron, manganese, molybdenum and zinc.

In one embodiment the composition of the subject invention is supported in or on a carrier. The carrier can be made of materials that can retain microorganisms thereon relatively loosely and thus facilitate easy release of microorganisms. The carrier is preferably inexpensive and can act as a nutrient source for the microorganisms thus applied, preferably a nutrient source that can be gradually released. Biodegradable carrier materials include, for example, cornhusk, sugar industry waste, or any agricultural waste (e.g., crop residue).

In certain embodiments, the composition comprises more than one microorganism that are compatible with one another. For example, strain MEC14XN can be used with another Meyerozyma sp. In one embodiment, the additional microorganism is Wickerhamomyces anomalus, e.g., W. anomalus NRRL Y-68030. Advantageously, W. anomalus NRRL Y-68030 is capable of solubilizing phosphorous and/or potassium into plant-bioavailable forms.

In one embodiment, B. subtilis NRRL B-68031 can be used in combination with B. amyloliquefaciens NRRL B-67928.

In certain embodiments, the composition further comprises one or more biosurfactants. The biosurfactants can be added in purified form, or crude form comprising, for example, a supernatant resulting from cultivation of a biosurfactant-producing microorganism. The crude form can optionally comprise residual nutrients, other microbial growth by-products, microorganisms and/or cellular components, in addition to the biosurfactant. In some embodiments, the biosurfactants are produced by the microbe(s) of the microbial fertilizer composition.

The biosurfactants can be selected from, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, and high molecular weight polymers such as lipoproteins, lipopolysaccharide-protein complexes, and polysaccharide-protein-fatty acid complexes.

The microbe-based composition can be formulated as, for example, a liquid suspension, an emulsion, a freeze- or spray-dried powder, pellets, granules, gels, tablets, capsules, and/or other forms, depending on mode of application and the target site.

The subject invention further provides methods of enhancing plant health, growth and/or yields, wherein the methods comprise applying an effective amount of a microbial fertilizer composition of the subject invention to the plant and/or to the soil in which the plant is growing or will be planted.

In certain embodiments, when live microorganisms are applied, the cells can grow in situ at the site of application and produce active compounds or growth by-products onsite. Consequently, a high concentration of microorganisms and beneficial growth by-products can be achieved easily and continuously at a treatment site.

To this end, the methods can comprise adding materials to enhance microbial growth during application (e.g., adding nutrients and/or prebiotics to promote microbial growth).

In one embodiment, by applying the subject composition directly onto the soil and either mixing the composition into the soil or allowing it to percolate into the soil, the method can be used to enrich the soil by replenishing it with plant-absorbable nitrogen stores (e.g., ammonia and/or nitrate). Advantageously, the methods can increase yields and enhance the quality of crops and produce due to the increase of organic nitrogen in the soil.

In certain embodiments, the microbial composition is applied alongside a nitrogen-based fertilizer, wherein the microorganism serves as an adjuvant or enhancer for the effects of the fertilizer. Advantageously, in some embodiments, this reduces the amount of fertilizer required to achieve a desired effect, reduces nitrous oxide emissions from soil, and/or reduces fertilizer runoff into water sources.

In certain embodiments, the microbial composition is applied during winter months and/or when temperatures are below 10° C. to enhance plant metabolic rates that would otherwise slow due to, for example, lack of available soil nutrition.

The compositions and methods utilize components that are biodegradable and toxicologically safe. Thus, the present invention can be used for enhancing production in agriculture, forestry, gardening, pasture restoration, and other plant-based applications as a “green” treatment, helping to reduce and/or eliminate the need for mineral fertilizers.

### BRIEF DESCRIPTION OF THE FIGURES

FIG. 1 shows sequence distribution of Meyerozyma sp. MEC14XN against Meyerozyma caribbica MG20W and Meyerozyma guilliermondii ATCC6260 genomes.

FIG. 2 shows nitrogen fixation screening results of various microorganisms using nitrogen-free (Burk's) agar plates.

FIG. 3 shows growth of the same microbes from FIG. 1 in nitrogen surplus media (TSA or PDA).

FIG. 4 shows nitrogen-solubilization measurements by Nessler's analysis for various microorganisms grown for 3 days in peptone water.

FIG. 5 shows nitrogen-solubilization measured by ammonium probe for highest performing microbes in the nitrogen-solubilization measurements of FIG. 4.

FIG. 6 shows nitrogen-fixation measured by Nessler's analysis for various microbes grown for 7 days in Burk's medium.

### DETAILED DESCRIPTION OF THE INVENTION

The present invention provides a microbial fertilizer comprising yeasts, bacteria and/or their growth by-products, which can be used for enhancing health, growth and/or yields of plants while replacing and/or reducing mineral fertilizer usage. Also provided are methods of obtaining and/or producing the microbial fertilizer composition.

### Selected Definitions

As used herein, “agriculture” means the cultivation and breeding of plants for food, fiber, biofuel, medicines, cosmetics, supplements, ornamental purposes and other uses. According to the subject invention, agriculture can also include horticulture, landscaping, gardening, plant conservation, forestry and reforestation, pasture and prairie restoration, orcharding, arboriculture, and agronomy. Further included in agriculture are the care, monitoring and maintenance of soil.

As used herein, a “broth,” “culture broth,” or “fermentation broth” refers to a culture medium comprising at least nutrients and microorganism cells.

Unless the context requires otherwise, the phrases “fermenting,” “fermentation process” or “fermentation reaction” and the like, as used herein, are intended to encompass both the growth phase and product biosynthesis phase of the process.

As used herein, “farmland” includes any tract of land in which plants are grown, cultivated and/or managed for human interests. Farmland includes:
pastures, or land containing mostly grasses, legumes and non-grass herbaceous plants, that is grazed by livestock;
meadows, which are typically ungrazed tracts of land that may be used for harvesting hay or other animal fodder;
rangelands, which include untended and human-tended grasslands, shrublands, woodlands, wetlands and deserts that are grazed by domestic livestock or wild animals; and
agricultural crops.

As used herein, an “isolated” or “purified” compound is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. “Isolated” in the context of a microbial strain means that the strain is removed from the environment in which it exists in nature. Thus, the isolated strain may exist as, for example, a biologically pure culture, or as spores (or other forms of the strain) in association with a carrier.

As used herein, a “biologically pure culture” is a culture that has been isolated from materials with which it is associated in nature. In a preferred embodiment, the culture has been isolated from all other living cells. In further preferred embodiments, the biologically pure culture has advantageous characteristics compared to a culture of the same microbe as it exists in nature. The advantageous characteristics can be, for example, enhanced production of one or more growth by-products.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 85%, 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, “enhancing” means improving or increasing. For example, enhanced plant health can mean improving the plant's ability grow and thrive, which includes increased seed germination and/or emergence, improved immunity against pests and/or diseases, and improved ability to survive environmental stressors, such as droughts and/or overwatering. Enhanced plant growth and/or enhanced plant biomass can mean increasing the size and/or mass of a plant above and/or below the ground (e.g., increased canopy/foliar volume, height, trunk caliper, branch length, shoot length, protein content, root size/density and/or overall growth index), and/or improving the ability of the plant to reach a desired size and/or mass. Enhanced yields can mean improving the end products produced by the plants in a crop, for example, by increasing the number and/or size of fruits, leaves, roots and/or tubers per plant, and/or improving the quality of the fruits, leaves, roots and/or tubers (e.g., improving taste, texture, brix, chlorophyll content and/or color).

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites include, but are not limited to, biosurfactants, biopolymers, enzymes, acids, solvents, alcohols, proteins, vitamins, minerals, microelements, and amino acids.

The subject invention utilizes “microbe-based compositions,” meaning a composition that comprises components that were produced as the result of the growth of microorganisms or other cell cultures. Thus, the microbe-based composition may comprise the microbes themselves and/or by-products of microbial growth. The microbes may be in a vegetative state, in spore or conidia form, in hyphae form, in any other form of propagule, or a mixture of these. The microbes may be planktonic or in a biofilm form, or a mixture of both. The by-products of growth may be, for example, metabolites, cell membrane components, proteins, and/or other cellular components. The microbes may be intact or lysed. In preferred embodiments, the microbes are present, with growth medium in which they were grown, in the microbe-based composition. The microbes may be present at, for example, a concentration of at least 1×10⁴, 1×10⁵, 1×10⁶, 1×10⁷, 1×10⁸, 1×10⁹, 1×10¹⁰, 1×10¹¹, 1×10¹² or 1×10¹³ or more CFU per gram or per ml of the composition.

The subject invention further provides “microbe-based products,” which are products that are to be applied in practice to achieve a desired result. The microbe-based product can be simply a microbe-based composition harvested from a microbe cultivation process. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “nitrogen fixation” or “fixation of atmospheric nitrogen” includes biological processes in which molecular nitrogen or nitrogen in the atmosphere (e.g., N₂) is converted into one or more nitrogenous compounds, including, but not limited to, ammonia, ammonium salts, urea, and nitrates.

As used herein “preventing” or “prevention” of a situation or occurrence means delaying, inhibiting, suppressing, forestalling, and/or minimizing the onset, extensiveness or progression of the situation or occurrence. Prevention can include, but does not require, indefinite, absolute or complete prevention, meaning it may still develop at a later time. Prevention can include reducing the severity of the onset of such a situation or occurrence, and/or stalling its development to a more severe or extensive situation or occurrence.

Ranges provided herein are understood to be shorthand for all of the values within the range. For example, a range of 1 to 20 is understood to include any number, combination of numbers, or sub-range from the group consisting of 1, 2, 3, 4, 5, 6, 7, 8, 9, 10, 11, 12, 13, 14, 15, 16, 17, 18, 19, 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduction” refers to a negative alteration, and the term “increase” refers to a positive alteration, wherein the negative or positive alteration is at least 0.25%, 0.5%, 1%, 5%, 10%, 15%, 20%, 25%, 30%, 35%, 40%, 45%, 50%, 55%, 60%, 70%, 75%, 80%, 85%, 90%, 95%, or 100%.

As used herein, “reference” refers to a standard or control condition.

As used herein, a “soil amendment” or a “soil conditioner” is any compound, material, or combination of compounds or materials that are added into soil to enhance the properties of the soil and/or rhizosphere. Soil amendments can include organic and inorganic matter, and can further include, for example, fertilizers, pesticides and/or herbicides. Nutrient-rich, well-draining soil is essential for the growth and health of plants, and thus, soil amendments can be used for enhancing the plant biomass by altering the nutrient and moisture content of soil. Soil amendments can also be used for improving many different qualities of soil, including but not limited to, soil structure (e.g., preventing compaction); improving the nutrient concentration and storage capabilities; improving water retention in dry soils; and improving drainage in waterlogged soils.

As used herein, “surfactant” refers to a compound that lowers the surface tension (or interfacial tension) between phases. Surfactants act as, e.g., detergents, wetting agents, emulsifiers, foaming agents, and dispersants. A “biosurfactant” is a surfactant produced by a living organism and/or using naturally-derived substrates.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof. All references cited herein are hereby incorporated by reference in their entirety.

### Microbial Fertilizers and Methods of Use

In preferred embodiments, a microbial fertilizer composition is provided comprising a yeast, bacterium and/or a growth by-product thereof, and optionally, a fermentation medium (e.g., broth or solid-state substrate) in which the microbe was cultivated.

Advantageously, the microbe-based compositions according to the subject invention are non-toxic and can be applied in high concentrations without causing irritation and/or toxicity to, for example, a human or animal's skin or digestive tract.

In one embodiment, the microbe(s) according to the subject invention are capable of fixing nitrogen.

In one embodiment, the microbe(s) according to the subject invention were not previously known to possess the ability to convert atmospheric nitrogen to nitrogenous compounds that can be used by plants as nutrients. In certain embodiments, the microorganism's genome comprises a gene or a cluster of genes encoding a nitrogen fixing mechanism, such as an enzyme and/or enzyme complex.

The microorganisms according to the systems and methods of the subject invention can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end. In certain preferred embodiments, the microorganism is not genetically modified.

In preferred embodiments, the microorganism is a yeast, for example, a Meyerozyma sp. a Wickerhamomyces sp., a Yarrowia sp., a Metschnikowia sp., or a Debaryomyces sp.

In other embodiments, the microorganism is a Bacillus sp. bacterium, such as B. subtilis or B. amyloliquefaciens.

Other non-limiting examples include Ascoidea rubescens, Brettanomyces bruxellensis, Brettanomyces naardenensis, Candida dubliniensis CD36, Candida intermedia, Candida maltosa Xu316, Candida viswanathii, Clavispora lusitaniae, Cyberlindnera jadinii NRRL Y-1542, Cyberlindnera fabianii, Debaryomyces hansenii CBS767, Geotrichum candidum, Kazachstania Africana, Kazachstania saulgeensis, Kuraishia capsulata, Lachancea dasiensis CBS 10888, Lachancea lanzarotensis, Lachancea meyersii CBS 8951, Lachancea mirantina, Lachancea nothofagi CBS 11611, Lachancea thermotolerans CBS 6340, Lachancea quebecensis, Lodderomyces elongisporus NRRL YB-4239, Metschnikowia sp. JCM 33374, Metschnikowia bicuspidate, Meyerozyma sp. JA9, Meyerozyma guilliermondii ATCC 6260, Millerozyma furinosa CBS 7064, Ogataea parapolymorpha, Pachysolen tannophilus NRRL Y-2460. Saccharomyces eubayanus, Saccharomycodes ludwigii, Spathaspora passalidarum NRRL Y-27907, Sugiyamaella lignohabitans, Suhomyces tanzawaensis NRRL Y-17324, Tetrapisispora blattae CBS 6284, Tetrapisispora phaffii CBS 4417, Trichomonascus ciferrii, Wickerhamiella sorbophila, Wickerhamomyces anomalus NRRL Y-68030 and Wickerhamomyces anomalus NRRL Y-366-8.

In a specific embodiment, the composition comprises Meyerozyma guilliermondii or Meyerozyma caribbica (e.g., M. caribbica subsp. Locus). In a specific preferred embodiment, the yeast is Meyerozyma sp. MEC14XN.

Advantageously, in certain embodiments, Meyerozyma sp. MEC14XN is more effective at fixing nitrogen than known nitrogen-fixing microorganisms, such as, e.g., B. amyloliquefaciens, Wickerhamomyces anomalus, and other strains of Meyerozyma. Other advantageous characteristics of this microbe include, for example, that it is endophytic and can provide nitrogen directly into plant roots; it can solubilize nitrogen; it is psycrophilic, capable of growing and reproducing in temperatures less than 10° C.′, more preferably less than 5° C.; it is salt-tolerant, capable of growing in high salinity soils; it is faster growing in soil and easier to obtain than traditional mycorrhizal fungi (which are mostly produced outside of the United States); it is capable of inhibiting plant-pathogenic Fusarium spp. fungi; and it is compatible with other plant-beneficial species of killer yeasts, such as Wickerhamomyces anomalus.

TABLE 1
Properties of MEC14XN

| Parameter | Test or Equipment | ME14 Results |
|---|---|---|
| Phosphorus (P) | Pikovskayas insoluble | Dissolved insoluble |
| solubilization | phosphorus plate test | phosphorus; |
| | | Solubilization index: 3.93 |
| | | at 8 days |
| Potassium (K) | Aleksandrov insoluble | Dissolved insoluble |
| solubilization | potassium plate test | potassium; |
| | | Solubilization index: 3.6 |
| | | at 14 days |
| Ammonia/Amine | Nessler's test in | 32.18 mg/L |
| Production | nitrogen free media | |
| (N-fixation) | | |
| Nitrogen | Nessler's test in | 201.3 mg/L |
| Solubilization | nitrogen surplus | |
| | media/ammonium | |
| | probe | |
| Growth | Potato Dextrose agar/ | Fast growth 25° C. to 42° C. |
| temperature | Tryptic Soy agar | Slow growth 4° C. |
| range | | No growth 45° C. |
| Salinity | Shaker flask growth | Good growth with 10% NaCl |
| tolerance | comparison using | minimal growth or no growth |
| | Varioskan | with 24% NaCl or above |
| Drought | Serial dilution plating | Freeze dried product is stable |
| tolerance | using PDA/TSA plate | at room temperature for |
| | at different storage | 4 weeks. |
| | time | |
| pH tolerance | Shaker flask growth | Can grow from pH 3.0 to 8.0 |
| | comparison using | Can survive at pH 2.0 without |
| | Varioskan | growth |

In certain embodiments, more than one yeast is used in the composition, for example, Meyerozyma sp. MEC14XN and Wickerhamomyces anomalus NRRL Y-68030.

A culture of the Wickerhamomyces anomalus NRRL Y-68030 microbe has been deposited with the Agricultural Research Service Northern Regional Research Laboratory (NRRL) Culture Collection, 1815 N. University St., Peoria, IL, USA. The deposit has been assigned accession number NRRL Y-68030 by the depository and was deposited on May 6, 2021.

In one embodiment, when Wickerhamomyces anomalus is utilized in the composition, the growth by-products can further comprise phytase, which is capable of converting phytate and/or phytic acid into plant-usable phosphorus.

In certain embodiments, the composition comprises one or more bacteria and/or growth by products thereof. The bacteria can be, for example, one or more Bacillus spp. bacteria. In certain embodiments, the Bacillus spp. are B. amyloliquefaciens (e.g., NRRL B-67928), B. subtilis (e.g., strain “B4” NRRL B-68031) and/or B. licheniformis. Bacteria can be used in spore form, as vegetative cells, and/or as a mixture thereof.

In certain embodiments, Bacillus subtilis B4 is used, having exemplary properties as outlined below.

TABLE 2
Properties of Bacillus subtilis B4

| Parameter | Test or Equipment | BSSL Results |
|---|---|---|
| Ammonia/Amine | Nessler's test in | 60.92 mg/L |
| Production | nitrogen | |
| (N-fixation) | free media | |
| Nitrogen | Nessler's test in | 156.50 mg/L |
| Solubilization | nitrogen surplus | |
| | media/ammonium probe | |
| Growth | Potato Dextrose agar/ | Fast growth 25° C. |
| temperature | Tryptic Soy agar | to 55° C. |
| range | | No growth 4° C. |
| | | No growth 60° C. |
| Drought | Serial dilution plating | Freeze dried product is |
| tolerance | using PDA/TSA plate at | stable at room temperature |
| | different storage time | for minimum 1 year |
| pH | Fermentation growth | Can grow between pH 5.0 |
| tolerance | comparison at different | to 8.0 Can survive at pH |
| | pH | 2.0 without growth |
| Biosurfactant | Continuous Fermentation | 8.87 g/L/d |
| Production | | |

A culture of the B4 microbe has been deposited with the Agricultural Research Service Northern Regional Research Laboratory (NRRL) Culture Collection, 1815 N. University St., Peoria, IL, USA. The deposit has been assigned accession number NRRL B-68031 by the depository and was deposited on May 6, 2021.

In one embodiment, the composition comprises B. amyloliquefaciens. In a preferred embodiment, the strain of B. amyloliquefaciens is B. amyloliquefaciens NRRL B-67928 (“B. amy”). A culture of the B. amyloliquefaciens “B. amy” microbe has been deposited with the Agricultural Research Service Northern Regional Research Laboratory (NRRL) Culture Collection, 1815 N. University St., Peoria, IL, USA. The deposit has been assigned accession number NRRL B-67928 by the depository and was deposited on Feb. 26, 2020.

Each of the subject cultures has been deposited under conditions that assure that access to the culture will be available during the pendency of this patent application to one determined by the Commissioner of Patents and Trademarks to be entitled thereto under 37 CFR 1.14 and 35 U.S.C 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

In one embodiment, the composition comprises at least about 1×10⁶ to 1×10¹², 1×10⁷ to 1×10¹¹, 1×10⁸ to 1×10¹⁰, or 1×10⁹ CFU per ml or gram of each microorganism. In one embodiment, the microorganisms of the subject composition comprise about 5 to 20% of the total composition by weight, or about 8 to 15%, or about 10 to 12%.

The composition can comprise the leftover fermentation substrate and/or purified or unpurified growth by-products, such as enzymes, biosurfactants and/or other metabolites. The microbes can be live or inactive.

The microbes and microbe-based compositions of the subject invention have a number of beneficial properties that are useful for improving crop production. For example, the compositions can comprise products resulting from the growth of the microorganisms, such as biosurfactants, proteins and/or enzymes, either in purified or crude form.

The microbes can also be useful for promoting increased above- and below-ground plant biomass per plant, increased high-carbon content polymers in plant tissue, increased numbers of plants per unit of area, increased uptake by microorganisms of organic compounds secreted by plants, reduced nitrogen-rich fertilizer usage, increased size and/or quantity of carbon- and water-binding soil-mineral aggregates, improved retention and dispersion of water in soil, reduced soil salinity and pollutant content, and increased microbial biomass and necromass in soil. Furthermore, in some embodiments, the microorganisms can induce auxin production, enable solubilization, absorption and/or dispersion of nutrients in the soil, and protect plants from pests and pathogens.

In certain embodiments, the composition comprises a growth by-product of the microbe, wherein the growth by-product is an enzyme capable of fixing nitrogen. In certain embodiments, the nitrogen fixing enzyme is a nitrogenase or an analog of a nitrogenase.

In some embodiments, a nitrogenase or analog thereof comprises an iron- and sulfur-containing co-factor that comprises a heterometal complex in the active site. In some species, this complex comprises a central molybdenum atom; however, in some other species it is replaced by a vanadium or iron atom.

In some embodiments, the enzyme is flavodoxin or an analog of flavodoxin. In some embodiments, flavodoxin is an analog of a nitrogenase.

An analog of a molecule does not have an identical chemical formula but can have similar structure and/or functions. A “structural analog” or “chemical analog” is a compound having a structure that is similar to that of another compound, but having one or more differing components, such as one or more different atoms, functional groups, or substructures. As used herein, “functional analogs,” are compounds that have similar physical, chemical, biochemical, or pharmacological properties. Despite their similarities, however, chemical analogs can be, but are not always, functional analogs, and functional analogs can be, but are not always, chemical analogs.

In some embodiments, the nitrogen fixing enzyme is an enzyme complex comprising a reductase and: a nitrogenase or analog thereof, or a flavodoxin or analog thereof.

In certain embodiments, the concentration of the nitrogen fixing enzyme in the composition is from 1 to 10,000 u/ml, from 100 to 9,000 u/ml, or from 200 to 8,000 u/ml.

In some embodiments, the composition further comprises ATP or another electron donor for facilitating the nitrogen fixation reaction.

In one embodiment, the microbial fertilizer composition is simply the fermentation medium (e.g., broth or solid-state substrate) containing the microorganism and/or the microbial metabolites produced by the microorganism and/or any residual nutrients. The product of fermentation may be used directly without extraction or purification of the microorganism and/or its growth by-product(s). If desired, extraction and purification can be easily achieved using standard extraction and/or purification methods or techniques described in the literature.

The microorganisms in the microbial fertilizer composition may be in an active or inactive form. In some embodiments, the microbe-based products may contain combinations of active and inactive microorganisms. In some embodiments, the composition does not contain microorganisms.

In certain embodiments, the microbe-based composition of the subject invention can comprise fermentation broth containing a microbial culture and/or the microbial metabolites produced by the microorganism and/or any residual nutrients. The composition may be, for example, at least, by volume, 1%, 5%, 10%, 25%, 50%, 75%, or 100% broth. The amount of biomass in the composition, by weight, may be, for example, anywhere from 0% to 100% inclusive of all percentages therebetween, or, for example from 5 g/l to 180 g/l or more, or from 10 g/l to 150 g/l.

The microbe-based products may be used without further stabilization, preservation, and storage. The microbes and/or broth resulting from the microbial growth can be removed from the growth vessel and transferred via, for example, piping for immediate use. Advantageously, direct usage of these microbe-based products preserves a high viability of the microorganisms, reduces the possibility of contamination from foreign agents and undesirable microorganisms, and maintains the activity of the by-products of microbial growth.

Alternatively, the microbes and/or broth can be harvested and transferred to containers for storage and/or for further processing.

For example, further components can be added as the harvested product is placed into containers and/or piped (or otherwise transported for use). The additives can be, for example, organic and/or inorganic materials, carriers, buffering agents, other microbe-based compositions produced at the same or different facility, adjuvants, viscosity modifiers, preservatives, nutrients for microbe growth, tracking agents, biocides, non-biological surfactants, emulsifying agents, lubricants, buffering agents, solubility controlling agents, pH adjusting agents, stabilizers, ultra-violet light resistant agents and other ingredients specific for an intended use.

In one embodiment, the composition further comprises one or more sources of nutrients for the microbe and/or for plants, such as, an organic substrate component, and/or an inorganic substrate component. The organic substrate component can comprise a carbon source or other nutrients for the microbial cells in the composition, for example, coal-mine waste, weathered coal, vegetable oil, glycerol, glucose, molasses, kelp extract, humic acid, fulvic acid, bone meal, blood meal, compost, corn gluten, potash, manure from a variety of animals including horses, cows, pigs, chickens, and/or sheep, or any materials that contain more than 20% of organic substances. Combinations and mixtures of such organic materials can also be used. Organic compounds present in such materials are decomposed by the microbe capable of breaking complex or high molecular weight carbon-chain molecules into simple carbon compounds so that they can be used by plants and other microbial cells in the fertilizer.

The inorganic substrate component can provide the microbial cells with minerals, materials and compounds containing, for example, nitrogen, phosphorus and/or potassium (e.g., NPK fertilizers, phosphate rock and/or potassium mica). The organic and inorganic substrate components may also provide the plants with other minerals such as, but not limited to, calcium magnesium and sulfur; and micronutrients, such as but not limited to, boron, copper, iron, manganese, molybdenum and zinc.

The organic and inorganic materials used in the invention should not contain amounts of toxic substances or microorganisms that can inhibit the growth of the microbial cells or plants.

The organic and inorganic components in the present invention are ground into suitable forms and sizes before incorporated into the fertilizer. Typically, the organic or inorganic material is conveyed into a crusher where it is broken up into pieces of ≤5 cm in diameter. Any conventional crusher or equivalent machines can be used for this purpose. The pieces are then transferred to a grinder by any conveying means and ground to a powder of ≥150 mesh. Any grinder that allows fine grinding can be used for this purpose. The powder is then conveyed to an appropriate storage tank for storage until use with other components of the fertilizer.

In one embodiment the composition of the subject invention comprises and/or is supported in or on a carrier. The carrier can be made of materials that can retain microorganisms thereon relatively mildly and thus allow easy release of microorganisms thus proliferated. The carrier can act as a nutrient source for the microorganisms thus applied, particularly a nutrient source that can be gradually released. Carriers can be comprised of solid-based, dry materials for formulation into tablet, capsule, granule or powder form; or the carrier can be comprised of liquid or gel-based materials for formulations into liquid or gel forms. For example, carriers can comprise water, saline, biopolymers, natural plant fibers, materials such as clay, silage, vermiculite, pumice, or paper sludge. A biodegradable carrier can also be used, for example, cornhusk, sugar industry waste, or any agricultural waste (e.g., crop residue).

To improve or stabilize the effects of the composition, it can be blended with suitable adjuvants and then used as such or after dilution, if necessary. In preferred embodiments, the composition is formulated as a liquid, a concentrated liquid, or as dry powder or granules that can be mixed with water and other components to form a liquid product.

In one embodiment, the composition can comprise glucose (e.g., in the form of molasses), glycerol and/or glycerin, as, or in addition to, an osmoticum substance, to promote osmotic pressure during storage and transport of the dry product.

In some embodiments, the composition further comprises additional crude form or purified microbial growth-products, such as biosurfactants, solvents, acids, proteins, minerals, vitamins and/or other enzymes. Crude form metabolites can comprise, for example, fermentation broth, cells, residual nutrients, and/or residual nutrients resulting from cultivation of a microbe, in addition to the metabolite. This crude form solution can comprise from about 25% to about 75%, from about 30% to about 70%, from about 35% to about 65%, from about 40% to about 60%, from about 45% to about 55%, or about 50% pure metabolite.

In certain embodiments, the composition further comprises one or more biosurfactants. The biosurfactants can be added in purified form, or crude form comprising a supernatant resulting from cultivation of a biosurfactant-producing microorganism. The crude form can optionally comprise residual nutrients, other microbial growth by-products, microorganisms and/or cellular components, in addition to the biosurfactant. In some embodiments, the biosurfactants are produced by the microorganism(s) of the microbial fertilizer composition.

Biosurfactants can be useful for enhancing the wettability and/or drainage of soils, which can be useful for enhancing the mobility of metals and other nutrient compounds in soil to increase their bioavailability to plant roots. Thus, in certain embodiments, the presence of biosurfactants in the composition can help enhance the fertility of soils, particularly with respect to nitrogen uptake by plant roots.

In some embodiments, the biosurfactants are selected from, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, and high molecular weight polymers such as lipoproteins, lipopolysaccharide-protein complexes, and polysaccharide-protein-fatty acid complexes. In one specific embodiment, the biosurfactant is glycolipid.

In certain embodiments, the concentration of the one or more biosurfactants in the composition is 0.001 to 90 by weight % (wt %), preferably 0.01 to 50 wt %, and more preferably 0.1 to 20 wt %.

In one embodiment, the composition may further comprise buffering agents including organic and amino acids or their salts, to stabilize pH near a preferred value. The pH of the microbe-based composition should be suitable for the microorganism of interest.

Suitable buffers include, but are not limited to, citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and mixtures thereof. Phosphoric and phosphorus acids or their salts may also be used. Synthetic buffers are suitable to be used but it is preferable to use natural buffers such as organic and amino acids or their salts.

In a further embodiment, pH adjusting agents include potassium hydroxide, ammonium hydroxide, potassium carbonate or bicarbonate, hydrochloric acid, nitric acid, sulfuric acid and mixtures thereof.

The pH of the composition should be suitable for the microorganism of interest as well as for the soil environment to which it will be applied. In some embodiments, the pH is about 2.0 to about 10.0, about 2.0 to about 9.5, about 2.0 to about 9.0, about 2.0 to about 8.5, about 2.0 to about 8.0, about 2.0 to about 7.5, about 2.0 to about 7.0, about 3.0 to about 7.5, about 4.0 to about 7.5, about 5.0 to about 7.5, about 5.5 to about 7.0, about 6.5 to about 7.5, about 3.0 to about 5.5, about 3.25 to about 4.0, or about 3.5. Buffers, and pH regulators, such as carbonates and phosphates, may be used to stabilize pH near a preferred value.

In one embodiment, additional components such as an aqueous preparation of a salt, such as sodium bicarbonate or carbonate, sodium sulfate, sodium phosphate, or sodium biphosphate, can be included in the microbe-based composition.

Advantageously, the microbe-based product may comprise fermentation medium (e.g., broth and/or solid-state substrate) in which the microbes were grown. The product may be, for example, at least, by weight or by volume, 1%, 5%, 10%, 25%, 50%, 75%, or 100% fermentation medium. The amount of biomass in the product, by weight or by volume, may be, for example, 0% to 100%. 1% to 95%, 5% to 80%, 10% to 65%, or 15% to 50%.

The compositions can be used either alone or in combination with other compounds and/or methods for efficiently enhancing plant health, growth and/or yields, and/or for supplementing the growth of the microorganisms. For example, in one embodiment, the composition can include and/or can be applied concurrently with nutrients and/or micronutrients for enhancing plant and/or microbe growth, such as magnesium, phosphate, nitrogen, potassium, selenium, calcium, sulfur, iron, copper, and zinc; and/or one or more prebiotics, such as kelp extract, fulvic acid, chitin, humate and/or humic acid. The exact materials and the quantities thereof can be determined by a grower or an agricultural scientist having the benefit of the subject disclosure.

The compositions can also be used in combination with other agricultural compounds and/or crop management systems. In one embodiment, the composition can optionally comprise, or be applied with, for example, natural and/or chemical pesticides, repellants, herbicides, fertilizers, water treatments, non-ionic surfactants and/or soil amendments.

If the composition is mixed with compatible chemical additives, the chemicals are preferably diluted with water prior to addition of the subject composition.

In certain embodiments, the microbe-based products of the subject invention have advantages over, for example, purified microbial metabolites alone, due to, for example, the use of the entire microbial culture. These advantages include one or more of the following: high concentrations of mannoprotein as a part of a yeast cell wall's outer surface; the presence of beta-glucan in yeast cell walls; the presence of enzymes and/or biosurfactants in the culture; and the presence of solvents and other metabolites in the culture. These advantages can be present when using active or inactive yeast.

In one embodiment, the composition is preferably formulated for application to soil, seeds, whole plants, or plant parts (including, but not limited to, roots, tubers, stems, flowers and leaves). In certain embodiments, the composition is formulated as, for example, liquid, dust, granules, microgranules, pellets, a gel, wettable powder, flowable powder, emulsions, microcapsules, oils, or aerosols.

Optionally, the composition can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

### Modes of Application

As used herein, “applying” a composition or product to a site refers to contacting a composition or product with a site such that the composition or product can have an effect on that site. The effect can be due to, for example, microbial growth and colonization, and/or the action of a metabolite, enzyme, biosurfactant or other microbial growth by-product, and/or activity of an accelerator substance. The mode of application depends upon the formulation of the composition, and can include, for example, spraying, pouring, sprinkling, injecting, spreading, mixing, dunking, fogging and misting. Formulations can include, for example, liquids, dry and/or wettable powders, flowable powders, dusts, granules, pellets, emulsions, microcapsules, steaks, oils, gels, pastes and/or aerosols. In an exemplary embodiment, the subject soil treatment composition is applied after the composition has been prepared by, for example, dissolving the composition in water.

In one embodiment, the site to which the composition is applied is the soil (or rhizosphere) in which plants will be planted or are growing (e.g., a crop, a field, an orchard, a grove, a pasture/prairie or a forest). The compositions of the subject invention can be pre-mixed with irrigation fluids, wherein the compositions percolate through the soil and can be delivered to, for example, the roots of plants to influence the root microbiome.

In one embodiment, the compositions are applied to soil surfaces, with or without water, where the beneficial effect of the soil application can be activated by rainfall, sprinkler, flood, or drip irrigation.

In one embodiment, the composition is applied to a plant or plant part. The composition can be applied directly thereto as a seed treatment, or to the surface of a plant or plant part (e.g., to the surface of the roots, tubers, stems, flowers, leaves, fruit, or flowers). In a specific embodiment, the composition is contacted with one or more roots of the plant. The composition can be applied directly to the roots, e.g., by spraying or dunking the roots, and/or indirectly, e.g., by administering the composition to the soil in which the plant grows (or the rhizosphere). The composition can be applied to the seeds of the plant prior to or at the time of planting, or to any other part of the plant and/or its surrounding environment.

In one embodiment, wherein the method is used in a large scale setting, such as in a crop, a muck field, a citrus grove, a pasture or prairie, a forest, a sod or turf farm, or another agricultural crop, the method can comprise administering the composition into a tank connected to an irrigation system used for supplying water, fertilizers, pesticides or other liquid compositions. Thus, the plant and/or soil surrounding the plant can be treated with the composition via, for example, soil injection, soil drenching, using a center pivot irrigation system, with a spray over the seed furrow, with micro-jets, with drench sprayers, with boom sprayers, with sprinklers and/or with drip irrigators. Advantageously, the method is suitable for treating hundreds or more acres of land.

In one embodiment, wherein the method is used in a smaller scale setting, the method can comprise pouring the composition (mixed with water and other optional additives) into the tank of a handheld lawn and garden sprayer and spraying soil or another site with the composition. The composition can also be mixed into a standard handheld watering can and poured onto a site.

Soil, plants and/or their environments can be treated at any point during the process of cultivating the plant. For example, the composition can be applied to the soil prior to, concurrently with, or after the time when seeds or plants are planted therein. It can also be applied at any point thereafter during the development and growth of the plant, including when the plant is flowering, fruiting, and during and/or after abscission of leaves.

### Crop Management

The subject compositions and methods can be useful for enhancing plant health, growth and/or yields, including that of livestock feed crops; enhancing sequestration of carbon in soil, vegetation and microbial biomass; and/or reducing fertilizer usage.

In one embodiment, methods are provided for enhancing plant health, growth and/or yields wherein one or more microorganisms is contacted with the plant and/or its surrounding environment. The method can comprise applying a soil treatment composition of the subject invention.

In certain embodiments, the microorganisms of the composition work synergistically with one another to enhance health, growth and/or yields in plants.

In one embodiment, the method can enhance plant health, growth and/or yields by enhancing root health and growth. More specifically, in one embodiment, the methods can be used to improve the properties of the rhizosphere in which a plant's roots are growing, for example, the nutrient and/or moisture retention and dispersion properties.

Additionally, in one embodiment, the method can be used to inoculate a plant's rhizosphere with one or more beneficial microorganisms. For example, in preferred embodiments, the microbes of the soil treatment composition can colonize the rhizosphere and provide multiple benefits to the plants that result in enhanced utilization and storage of carbon via enhanced growth and/or health of both aerial and subterranean plant tissue.

In some embodiments, the subject methods increase the above- and below-ground biomass of plants, which includes, for example, increased foliage volume, increased stem and/or trunk diameter, enhanced root growth and/or density, and/or increased total numbers of plants

In certain embodiments, the subject compositions and methods can be used for improving agricultural fertilization practices. For example, in preferred embodiments, the microbes of the soil treatment composition can colonize the rhizosphere and/or work synergistically with other free-living and endophytic nutrient-fixing microbes to provide enhanced solubilization of nutrients in the soil, such that the nutrients are more bioavailable for plant root uptake.

In some embodiments, the subject invention can be used to reduce and/or replace a chemical or synthetic fertilizer, wherein the composition comprises a microorganism capable of fixing, solubilizing, mobilizing and/or increasing the bioavailability and/or root uptake of nitrogen, potassium, K₂O, phosphorous, P₂O₅, and/or other micronutrients in soil, such as, e.g., S, Zn, B, and Mn. In other words, the subject invention can be useful for improving nutrient use efficiency and/or treating/preventing plant nutrient deficiencies.

In one embodiment, the method can be used to provide the plant with phosphorus in the form of phosphates. In certain embodiments, Wickerhamomyces anomalus can produce phytase, an enzyme that is capable of converting phytic acid present in soil into plant-bioavailable (e.g., root-absorbable) phosphates. Accordingly, the method can be used to treat and/or prevent a phosphorus deficiency in a plant, reduce phosphorus usage in fertilizers, and/or reduce phosphorus runoff into water sources.

In one embodiment, the method can be used for improving nitrogen use efficiency, as well as reducing nitrous oxide emissions. In some embodiments, the subject soil treatment compositions can be used synergistically with free-living and/or endophytic nitrogen fixers, such as Klebsiella, Azotobacter and/or Azospirillum, for microbial facilitated nutrient release and cycling. Thus, in some embodiments, improved nitrogen use efficiency, reduced nitrous oxide emissions, and reduced nitrogen runoff into water sources can be achieved by replacing some or all nitrogen-rich fertilizers and/or increasing soil nitrogen uptake by plant roots using soil treatment compositions according to the subject invention.

Advantageously, in certain embodiments, the subject methods can be used to enhance health, growth and/or yields in plants having compromised immune health due to an infection from a pathogenic agent or from an environmental stressor, such as, for example, drought. Thus, in certain embodiments, the subject methods can also be used for improving the immune health, or immune response, of plants.

For example, the plant may be affected by a pathogenic strain of Pseudomonas (e.g., P. savastanoi, P. syringae pathovars); Ralstonia solanacearum; Agrobacterium (e.g., A. tumefaciens); Xanthomonas (e.g., X. oryzae pv. Oryzae, X. campestris pathovars, X. axonopodis pathovars); Erwinia (e.g., E. amylovora); Xylella (e.g., X. fastidiosa); Dickeya (e.g., D. dadantii and D. solani); Pectobacterium (e.g., P. carotovorum and P. atrosepticum); Clavibacter (e.g., C. michiganensis and C. sepedonicus); Candidatus liberibacter asiaticus; Pantoea; Burkholderia; Acidovorax; Streptomyces; Spiroplasma; and/or Phytoplasma; as well as huanglongbing (HLB, citrus greening disease), citrus canker disease, citrus bacterial spot disease, citrus variegated chlorosis, brown rot, citrus root rot, citrus and black spot disease.

In one embodiment, the methods are used to enhance the health, growth and/or yields of citrus trees affected by citrus greening disease and/or citrus canker disease.

The present invention can be used to enhance health, growth and/or yields of plants and/or crops in, for example, agriculture, horticulture, greenhouses, landscaping, and the like. The present invention can also be used for improving one or more qualities of soil, thereby enhancing the performance of the soils for agricultural, home and gardening purposes. Furthermore, the present invention can be used in pasture management, as well as in professional turf, ornamental and landscape management.

In certain embodiments, the soil treatment composition may also be applied so as to promote colonization of the roots and/or rhizosphere as well as the vascular system of the plant in order to enhance plant health and vitality. The microbe-based product can also support a plant's vascular system by, for example, entering and colonizing said vascular system and contributing metabolites, and nutrients important to plant health and productivity.

In yet another embodiment, the method can be used to fight off and/or discourage colonization of the rhizosphere by soil microorganisms that are deleterious or that might compete with beneficial soil microorganisms.

In one embodiment, the method can be used for enhancing penetration of beneficial molecules through the outer layers of root cells, for example, at the root-soil interface of the rhizosphere.

In certain embodiments, this is achieved via the presence of a biosurfactant, either produced in situ by a microorganism of the subject soil treatment composition, or applied directly to the soil. Biosurfactants according to the subject invention can help reduce the surface tension of water, allowing for increased uptake of nutrients and water into the plant roots, as well as increased efficiency when circulating water and nutrients throughout the plant's extremities. This is useful even for tall trees that must fight gravity, as well as plants that might be diseased and experience vascular constriction due to, e.g., pathogenic biofilms. Furthermore, this mode of action can enhance not only the transport of beneficial materials into a plant, but also the exports of toxins, free radicals and waste products from the plant with more efficiency.

In certain embodiments, the subject biosurfactants are particularly useful for these purposes due to their small micelle size. In certain embodiments, a sophorolipid biosurfactant has a micelle size less than 50 nm, more preferably less than 25 nm. Advantageously, this allows for enhanced penetration of small spaces and/or pores, such as those found between cells and in biofilm matrices.

In certain embodiments, the subject method enhances plant utilization and storage of carbon, which is achieved by enhancing the accumulation of degradation-resistant organic polymers in plant tissue and/or soil. In some embodiments, the accumulation of degradation-resistant organic polymers is further enhanced by utilizing plants that have been modified to produce greater-than-normal amounts of a particular degradation-resistant organic polymer.

In certain embodiments, the degradation-resistant organic polymers are polysaccharides, polyaromatics and/or polyesters. Examples found in plants include, but are not limited to, suberin, cutin, cutan, and lignins. In preferred embodiments, due to, for example, the complex nature of their chemical structure, the degradation-resistant organic polymers are not readily biodegradable for, e.g., at least 1 year, at least 5 years, at least 25 years, at least 100 years, or even at least 1,000 years after the death of the plant.

In addition to enhancing plant utilization and storage of carbon, colonization of the roots and/or soil by the microbes of the subject composition can also increase soil carbon sequestration. In certain embodiments, increasing soil carbon sequestration is achieved by enhancing the growth of plant roots in the soil and/or increasing accumulation of degradation-resistant organic polymers in the plant roots.

In one embodiment, the methods and compositions according to the subject invention lead to an increase in one or more of: root length, root density, root mass, stalk diameter, plant height, canopy density, chlorophyll content, flower count, bud count, bud size, bud density, leaf surface area, oil content, fruit count, fruit size, fiber content, and/or nutrient uptake of a plant, by at least 1%, 5%, 10%, 20%, 30%, 40%, 50%, 60%, 70%, 80%, 90%, 100%, 150%, 200%, or more, compared to a plant growing in an untreated environment.

Reducing the Carbon Footprint and/or Carbon Intensity

A “carbon footprint” may be defined as a measure of the total amount of carbon dioxide (CO₂) and other GHGs emitted directly or indirectly by a human activity or accumulated over the full life cycle of a product or service. As just one example, a product that requires transportation over many miles by truck (e.g., harvested feed grains) may have a larger carbon footprint than an alternative product that does not require transportation (e.g., grass growing in a pasture).

Carbon footprints can be calculated using a Life Cycle Assessment (LCA) method, or can be restricted to the immediately attributable emissions from energy use of fossil fuels. A life cycle assessment (LCA, also known as life cycle analysis, ecobalance, and cradle-to-grave analysis) is the investigation and valuation of the environmental impacts of a given product or service caused or necessitated by its existence. The life cycle concept of the carbon footprint means that it is all-encompassing and includes all possible causes that give rise to carbon emissions. In other words, all direct (on-site, internal) and indirect emissions (off-site, external, embodied, upstream, downstream) need to be taken into account.

Normally, a carbon footprint is expressed as a CO₂ equivalent. Carbon dioxide equivalency is a quantity that describes, for a given mixture and amount of GHG, the amount of CO₂ that would have the same global warming potential (GWP), when measured over a specified timescale (generally, 100 years). Carbon dioxide equivalency thus reflects time-integrated radiative forcing. The carbon dioxide equivalency for a gas is obtained by multiplying the mass and the GWP of the gas. The following units are commonly used:
a) By the UN climate change panel IPCC: billion metric tonnes of CO₂ equivalent (GtCO₂ eq);
b) In industry: million metric tonnes of carbon dioxide equivalents (MMTCDE);
c) For vehicles: g of carbon dioxide equivalents/km (gCDE/km).

For example, the GWP for methane is 21 and for nitrous oxide 310. This means that emissions of 1 million metric tonnes of methane and nitrous oxide respectively is equivalent to emissions of 21 and 310 million metric tonnes of carbon dioxide.

Various methods exist in the art for calculating or estimating carbon footprints and may be employed in the subject invention.

Advantageously, in preferred embodiments, the subject invention can be useful for reducing the carbon footprint of agriculture.

A “reduced carbon footprint” means a negative alteration in the amount of carbon dioxide and other GHGs emitted per unit time over the full life cycle of producing crops, through and until an agricultural product is ultimately consumed by human consumers. The negative alteration in CO₂ and/or other GHG emissions can be, for example, at least 0.25%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

In some embodiments, the term “carbon footprint” is interchangeable herein with the terms “carbon intensity” and “emission intensity.” Emission intensity is the measure of the emission rate of a given GHG relative to the “intensity” of a specific activity or industrial process (e.g., burning of fuel, production of livestock animals, production of corn). The emissions intensity can include amount of emissions relative to, for example, amount of fuel combusted, yield of corn harvested, number of livestock animals produced, amount of an commercial product produced, total distance traveled, and/or number of economic units generated.

Emissions intensity is measured across the entire life cycle of a product. For example, the emissions intensity of fuels is calculated by compiling all of the GHG emissions emitted along the supply chain for a fuel, including all the emissions emitted in exploration, mining, collecting, producing, transporting, distributing, dispensing and burning the fuel.

In addition to reducing the carbon footprint and/or carbon intensity of agriculture and livestock production, in some embodiments, the subject invention can be used for reducing the number of carbon credits used by an operator involved in, e.g., agriculture, livestock production, forestry/reforestation, and wetland management.

Advantageously, the systems of the subject invention can increase the efficiency and reduce the financial and environmental costs of agricultural practices. In particular, the compositions and methods utilized according to the subject invention can help in preserving valuable natural resources, such as soil and water, while improving production of valuable plant and animal-based commodities.

### Target Plants

As used here, the term “plant” includes, but is not limited to, any species of woody, ornamental or decorative, crop or cereal, fruit plant or vegetable plant, flower or tree, macroalga or microalga, phytoplankton and photosynthetic algae (e.g., green algae Chlamydomonas reinhardtii). “Plant” also includes a unicellular plant (e.g., microalga) and a plurality of plant cells that are largely differentiated into a colony (e.g., volvox) or a structure that is present at any stage of a plant's development. Such structures include, but are not limited to, a fruit, a seed, a shoot, a stem, a leaf, a root, a flower petal, etc. Plants can be standing alone, for example, in a garden, or can be one of many plants, for example, as part of an orchard, crop or pasture.

As used herein, “crop plants” refer to any species of plant or alga, grown for profit and/or for sustenance for humans, animals or aquatic organisms, or used by humans (e.g., textile, cosmetics, and/or drug production), or viewed by humans for pleasure (e.g., flowers or shrubs in landscaping or gardens) or any plant or alga, or a part thereof, used in industry, commerce or education. Crop plants can be plants that can be obtained by traditional breeding and optimization methods or by biotechnological and recombinant methods, or combinations of these methods, including the transgenic plants and the plant varieties.

Types of crop plants that can benefit from application of the products and methods of the subject invention include, but are not limited to: row crops (e.g., corn, soy, sorghum, peanuts, potatoes, etc.), field crops (e.g., alfalfa, wheat, grains, etc.), tree crops (e.g., walnuts, almonds, pecans, hazelnuts, pistachios, etc.), citrus crops (e.g., orange, lemon, grapefruit, etc.), fruit crops (e.g., apples, pears, strawberries, blueberries, blackberries, etc.), turf crops (e.g., sod), ornamentals crops (e.g., flowers, vines, etc.), vegetables (e.g., tomatoes, carrots, etc.), vine crops (e.g., grapes, etc.), forestry (e.g., pine, spruce, eucalyptus, poplar, etc.), managed pastures (any mix of plants used to support grazing animals).

Additional examples of plants for which the subject invention is useful include, but are not limited to, cereals and grasses (e.g., wheat, barley, rye, oats, rice, maize, sorghum, corn), beets (e.g., sugar or fodder beets); fruit (e.g., grapes, strawberries, raspberries, blackberries, pomaceous fruit, stone fruit, soft fruit, apples, pears, plums, peaches, almonds, cherries or berries); leguminous crops (e.g., beans, lentils, peas or soya); oil crops (e.g., oilseed rape, mustard, poppies, olives, sunflowers, coconut, castor, cocoa or ground nuts); cucurbits (e.g., pumpkins, cucumbers, squash or melons); fiber plants (e.g., cotton, flax, hemp or jute); citrus fruit (e.g., oranges, lemons, grapefruit or tangerines); vegetables (e.g., spinach, lettuce, asparagus, cabbages, carrots, onions, tomatoes, potatoes or bell peppers); Lauraceae (e.g., avocado, Cinnamonium or camphor); and also tobacco, nuts, herbs, spices, medicinal plants, coffee, eggplants, sugarcane, tea, pepper, grapevines, hops, the plantain family, latex plants, cut flowers and ornamentals.

In certain embodiments, the crop plant is a citrus plant. Examples of citrus plants according to the subject invention include, but are not limited to, orange trees, lemon trees, lime trees and grapefruit trees. Other examples include Citrus maxima (Pomelo), Citrus medica (Citron), Citrus micrantha (Papeda), Citrus reticulata (Mandarin orange), Citrus paradisi (grapefruit), Citrus japonica (kumquat), Citrus australasica (Australian Finger Lime), Citrus australis (Australian Round lime), Citrus glauca (Australian Desert Lime), Citrus garrawayae (Mount White Lime), Citrus gracilis (Kakadu Lime or Humpty Doo Lime), Citrus inodora (Russel River Lime), Citrus warburgiana (New Guinea Wild Lime), Citrus wintersii (Brown River Finger Lime), Citrus halimii (limau kadangsa, limau kedut kera), Citrus indica (Indian wild orange), Citrus macroptera, and Citrus latipes, Citrus x aurantiifolia (Key lime), Citrus x aurantium (Bitter orange), Citrus x latifolia (Persian lime), Citrus x limon (Lemon), Citrus x limonia (Rangpur), Citrus x sinensis (Sweet orange), Citrus x tangerina (Tangerine), Imperial lemon, tangelo, orangelo, tangor, kinnow, kiyomi, Minneola tangelo, oroblanco, ugli, Buddha's hand, citron, bergamot orange, blood orange, calamondin, clementine, Meyer lemon, and yuzu.

In some embodiments, the crop plant is a relative of a citrus plant, such as orange jasmine, limeberry, and trifoliate orange (Citrus trifolata).

Additional examples of target plants include all plants that belong to the superfamily Viridiplantae, in particular monocotyledonous and dicotyledonous plants including fodder or forage legumes, ornamental plants, food crops, trees or shrubs selected from Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g., A. sativa, A. fatua, A. byzantina, A. fatua var. sativa, A. hybrida), Averrhoa carambola, Bambusa sp., Benincasa hispida, Bertholletia excelsea, Beta vulgaris, Brassica spp. (e.g., B. napus, B. rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g., E. guineensis, E. oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g., G. max, Soja hispida or Soja max), Gossypium hirsutum, Helianthus spp. (e.g., H. annuus), Hemerocallis fulva, Hibiscus spp., Hordeum spp. (e.g., H. vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g., L. esculentum, L. lycopersicum, L. pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g., O). sativa, O. latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp. (e.g., Q. suber L.), Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g., S. tuberosum, S. integrifolium or S. lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g., T. aestivum, T. durum, T. turgidum, T. hybernum, T. macha, T. sativum, T. monococcum or T. vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

Target plants can also include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Target vegetable plants include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the embodiments include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Plants of the embodiments include crop plants (for example, corn, alfalfa, sunflower, Brassica, soybean, cotton, safflower, peanut, sorghum, wheat, millet, tobacco, etc.), such as corn and soybean plants.

Target turfgrasses include, but are not limited to: annual bluegrass (Poa annua); annual ryegrass (Lolium multiflorum); Canada bluegrass (Poa compressa); Chewings fescue (Festuca rubra); colonial bentgrass (Agrostis tenuis); creeping bentgrass (Agrostis palustris); crested wheatgrass (Agropyron desertorum); fairway wheatgrass (Agropyron cristatum); hard fescue (Festuca longifolia); Kentucky bluegrass (Poa pratensis); orchardgrass (Dactylis glomerate); perennial ryegrass (Lolium perenne); red fescue (Festuca rubra); redtop (Agrostis alba); rough bluegrass (Poa trivialis); sheep fescue (Festuca ovine); smooth bromegrass (Bromus inermis); tall fescue (Festuca arundinacea); timothy (Phleum pretense); velvet bentgrass (Agrostis canine); weeping alkaligrass (Puccinellia distans); western wheatgrass (Agropyron smithii); Bermuda grass (Cynodon spp.); St. Augustine grass (Stenotaphrum secundatum); zoysia grass (Zoysia spp.); Bahia grass (Paspalum notatum); carpet grass (Axonopus affinis); centipede grass (Eremochloa ophiuroides); kikuyu grass (Pennisetum clandesinum); seashore paspalum (Paspalum vaginatum); blue gramma (Bouteloua gracilis); buffalo grass (Buchloe dactyloids); sideoats gramma (Bouteloua curtipendula).

Further plants of interest include grain plants that provide seeds of interest, oil-seed plants, and leguminous plants. Seeds of interest include grain seeds, such as corn, wheat, barley, rice, sorghum, rye, millet, etc. Oil-seed plants include cotton, soybean, safflower, sunflower, Brassica, maize, alfalfa, palm, coconut, flax, castor, olive etc. Leguminous plants include beans and peas. Beans include guar, locust bean, fenugreek, soybean, garden beans, cowpea, mungbean, lima bean, fava bean, lentils, chickpea, etc. Further plants of interest include Cannabis (e.g., sativa, indica, and ruderalis) and industrial hemp.

All plants and plant parts can be treated in accordance with the invention. In this context, plants are understood as meaning all plants and plant populations such as desired and undesired wild plants or crop plants (including naturally occurring crop plants). Crop plants can be plants that can be obtained by traditional breeding and optimization methods or by biotechnological and recombinant methods, or combinations of these methods, including the transgenic plants and the plant varieties.

Plant tissue and/or plant parts are understood as meaning all aerial and subterranean parts and organs of the plants such as shoots, leaves, flowers, roots, needles, stalks, stems, fruits, seeds, tubers and rhizomes. The plant parts also include crop material and vegetative and generative propagation material, for example cuttings, tubers, rhizomes, slips and seeds.

### Obtaining a Nitrogen-Fixing Microbe

In some embodiments, the ability of the microbe in the microbial fertilizer composition to fix nitrogen, or the level at which the yeast has enhanced ability to fix nitrogen, is non-natural, meaning a silent or weakly active biological mechanism within the microbe that is responsible for the ability to fix nitrogen has been activated and/or enhanced by certain influencing conditions during cultivation of the microbe.

In some embodiments, the biological mechanism is a gene or gene cluster, wherein the influencing conditions initiate an event that results in the activation and/or enhancement of an inactive (silent) or weakly active gene or gene cluster. Such activation and/or enhancement leads to an observable modulation in phenotype, e.g., transcription and translation into a protein, such as a nitrogen fixing enzyme. The influencing conditions may directly activate a gene or gene cluster, e.g., by directly activating the promoter, or may indirectly activate a gene or gene cluster, e.g., by activating other factors that act on the promoter.

In certain embodiments, the influencing conditions comprise an environmental and/or nutritional stressor. For example, in preferred embodiments, the influencing conditions comprise subjecting the microbe to cultivation in a nitrogen-free nutrient medium.

In preferred embodiments, a culture of a microbe possessing a nitrogen-fixation phenotype can be obtained utilizing clonal selection methods. In certain embodiments, the methods comprise identifying a microbe possessing a biological mechanism for producing a nitrogen fixing enzyme, wherein the biological mechanism is normally inactive (silent) or weakly active in the microbe, and subjecting the yeast strain to influencing conditions that activate and/or enhance the biological mechanism in the microbe strain.

In one embodiment, identification of a microbe that possesses a silent biological mechanism can be performed using a genetic search. For example, BLAST screening can be used to search the genomes of a plurality of microbe strains for gene orthologs or gene cluster orthologs of reference genes or gene clusters encoding nitrogen-fixing enzymes. Based on NCBI protocol for inferring homology from similarities between the microbe orthologs(s) and the reference gene(s), homology is inferred where a bit score of 50 or higher is obtained. (See Pearson 2013, incorporated by reference herein).

In preferred embodiments, subjecting the microbe strain to influencing conditions comprises:
a) inoculating a first nutrient medium containing no nitrogen with an inoculum culture of the identified microbe and leaving the inoculum culture in the first nutrient medium for an amount of time until a colony of the microbe grows;
b) collecting the colony; and
c) inoculating a second nutrient medium containing no nitrogen with the colony.

In one embodiment, a nitrogen-free medium comprises sources of carbon (e.g., sucrose, glucose, fructose, dextrose, maltose, xylose, starches, and the like), inorganic salts (e.g., CaCO3, KH2PO₄, MgSO4, NaCl, and CaSO4), amino acids, trace elements, agar and/or other non-nitrogen containing components that are consumable by microbes.

In one embodiment, the first nutrient medium and the second nutrient medium comprise the same components. In one embodiment, the first nutrient medium and second nutrient medium are held in separate containers, such as, e.g., separate petri dishes or well plates, wherein observation of colony growth and collection thereof can be performed readily.

In certain embodiments, the biological mechanism that is activated is a gene or a cluster of genes encoding a nitrogen fixing enzyme. Advantageously, in one embodiment, once the biological mechanism is activated in the microbe, it does not become inactivated.

In certain embodiments, the nitrogen fixing enzyme is a nitrogenase or an analog of a nitrogenase. In certain embodiments, the enzyme is a flavodoxin or an analog of a flavodoxin. In some embodiments, the flavodoxin is an analog to a nitrogenase.

In certain embodiments, the nitrogen fixing enzyme is an enzyme complex. For example, in certain embodiments, the activated biological mechanism is a nitrogen fixing enzyme complex comprising a reductase and: a nitrogenase or an analog thereof, or a flavodoxin or an analog thereof.

In one embodiment, the absence of nitrogen in the nutrient medium influences activation and/or enhancement of the biological mechanism in one or more microbe cells of the inoculum culture, wherein activation and/or enhancement of the biological mechanism enables the one or more microbe cells to unnaturally produce a nitrogen fixing enzyme and/or produce a nitrogen fixing enzyme in enhanced quantities.

Thus, in one embodiment, the microbe cells in which the biological mechanism is activated and/or enhanced (“activated microbe”) are able to survive by unnaturally utilizing atmospheric di-nitrogen as a nitrogen source. Without activation or enhancement of the biological mechanism, the microbe cells would not survive and/or grow due to a lack of usable nitrogen; thus, the influencing conditions trigger the activation and/or enhancement of the biological mechanism.

In some embodiments, steps a) through c) are repeated a plurality of times until an inoculum culture of a microbe strain that is entirely, or nearly entirely, activated is obtained. This can be determined as the point at which, for example, approximately 85% to 100% of the inoculum culture in a nutrient medium (e.g., 85% to 100% of the area within a petri dish) shows signs of colony growth after a certain time period.

In some embodiments, more than one colony can form in the nitrogen-free nutrient medium. Preferably, in such an instance, the colony that appeared first in time is collected for the next round of inoculation.

In one embodiment, each repetition results in a shorter amount of time required to influence activation of the biological mechanism, until the amount of time reaches less than 24 hours, preferably less than 12 hours. In one embodiment, each repetition results in greater numbers of colonies that are formed comprising activated microbe cells within a certain period of time, e.g., within 12 hours to 7 days of inoculation.

In some embodiments, the microbe culture is tested for the change in phenotype allowing it to fix nitrogen. The term “phenotype” denotes characteristics or traits of the recipient microorganism, such as its morphology, development, biochemical or physiological properties or behavior. Such characteristics or traits include the presence or absence or changed amounts of growth by-products (e.g., nitrogen fixing enzymes). For example, a modified acetylene reduction method for measuring nitrogen fixed by microbes can be used to test for such a change in phenotype. (See U.S. Pat. No. 5,578,486, incorporated by reference herein).

In one embodiment, the amount of nitrogen the microbe cells can fix is at least about 5 mg to 50 mg, or about 10 mg to 25 mg, for each gram of microbe dry weight.

### Growth of Microbes According to the Subject Invention

The subject invention utilizes methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites (e.g. small molecules and proteins), residual nutrients and/or intracellular components (e.g. enzymes and other proteins).

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of organisms in a sample. The technique can also provide an index by which different environments or treatments can be compared.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source can be a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sunflower oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In some embodiments, the method for cultivation may further comprise adding additional acids and/or antimicrobials in the medium before, and/or during the cultivation process. Antimicrobial agents or antibiotics are used for protecting the culture against contamination.

Additionally, antifoaming agents may also be added to prevent the formation and/or accumulation of foam during submerged cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

The pH of the culture should be suitable for the microorganism of interest as well as for the soil environment to which the composition will be applied. In some embodiments, the pH is about 2.0 to about 10.0, about 2.0 to about 9.5, about 2.0 to about 9.0, about 2.0 to about 8.5, about 2.0 to about 8.0, about 2.0 to about 7.5, about 2.0 to about 7.0, about 3.0 to about 7.5, about 4.0 to about 7.5, about 5.0 to about 7.5, about 5.5 to about 7.0, about 6.5 to about 7.5, about 3.0 to about 5.5, about 3.25 to about 4.0, or about 3.5. Buffers, and pH regulators, such as carbonates and phosphates, may be used to stabilize pH near a preferred value.

In one embodiment, the method of cultivation is carried out at about 5° to about 100° C., about 15° to about 60° C., about 20° to about 50° C., about 20° to about 45° C., about 25° to about 40° C., about 25° to about 37° C., about 25° to about 35° C., about 30° to about 35° C., about 24° to about 28° C., or about 22° to about 25° C. In one embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The microbial growth by-product produced by microorganisms of interest may be retained in the microorganisms or secreted into the growth medium. The medium may contain compounds that stabilize the activity of microbial growth by-product.

The biomass content of the fermentation medium may be, for example, from 5 g/l to 180 g/l or more, or from 10 g/l to 150 g/l.

The cell concentration may be, for example, at least 1×10⁶ to 1×10¹³, 1×10⁷ to 1×10¹², 1×10⁸ to 1×10¹¹, or 1×10⁹ to 1×10¹⁰ CFU/ml.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

Advantageously, the microbe-based products can be produced in remote locations. The microbe growth facilities may operate off the grid by utilizing, for example, solar, wind and/or hydroelectric power.

### Preparation of Microbe-Based Products

One microbe-based product of the subject invention is simply the fermentation medium containing the microorganisms and/or the microbial metabolites produced by the microorganisms and/or any residual nutrients. The product of fermentation may be used directly without extraction or purification. If desired, extraction and purification can be easily achieved using standard extraction and/or purification methods or techniques described in the literature.

The microorganisms in the microbe-based products may be in an active or inactive form, or in the form of vegetative cells, reproductive spores, conidia, mycelia, hyphae, or any other form of microbial propagule. The microbe-based products may also contain a combination of any of these forms of a microorganism.

In one embodiment, different strains of microbe are grown separately and then mixed together to produce the microbe-based product. The microbes can, optionally, be blended with the medium in which they are grown and dried prior to mixing.

In one embodiment, the different strains are not mixed together, but are applied to a plant and/or its environment as separate microbe-based products.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

Upon harvesting the microbe-based composition from the growth vessels, further components can be added as the harvested product is placed into containers or otherwise transported for use. The additives can be, for example, buffers, carriers, other microbe-based compositions produced at the same or different facility, viscosity modifiers, preservatives, nutrients for microbe growth, surfactants, emulsifying agents, lubricants, solubility controlling agents, tracking agents, solvents, biocides, antibiotics, pH adjusting agents, chelators, stabilizers, ultra-violet light resistant agents, other microbes and other suitable additives that are customarily used for such preparations.

In one embodiment, buffering agents including organic and amino acids or their salts, can be added. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

In one embodiment, additional components such as an aqueous preparation of a salt, such as sodium bicarbonate or carbonate, sodium sulfate, sodium phosphate, sodium biphosphate, can be included in the formulation.

In certain embodiments, an adherent substance can be added to the composition to prolong the adherence of the product to plant parts. Polymers, such as charged polymers, or polysaccharide-based substances can be used, for example, xanthan gum, guar gum, levan, xylinan, gellan gum, curdlan, pullulan, dextran and others.

In preferred embodiments, commercial grade xanthan gum is used as the adherent. The concentration of the gum should be selected based on the content of the gum in the commercial product. If the xanthan gum is highly pure, then 0.001% (w/v-xanthan gum/solution) is sufficient.

In one embodiment, glucose, glycerol and/or glycerin can be added to the microbe-based product to serve as, for example, an osmoticum during storage and transport. In one embodiment, molasses can be included.

In one embodiment, prebiotics can be added to and/or applied concurrently with the microbe-based product to enhance microbial growth. Suitable prebiotics, include, for example, kelp extract, fulvic acid, chitin, biochar, humate and/or humic acid. In a specific embodiment, the amount of prebiotics applied is about 0.1 L/acre to about 0.5 L/acre, or about 0.2 L/acre to about 0.4 L/acre.

In one embodiment, specific nutrients are added to and/or applied concurrently with the microbe-based product to enhance microbial inoculation and growth. These can include, for example, soluble potash (K₂O), magnesium, sulfur, boron, iron, manganese, and/or zinc. The nutrients can be derived from, for example, potassium hydroxide, magnesium sulfate, boric acid, ferrous sulfate, manganese sulfate, and/or zinc sulfate.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., a citrus grove). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This allows for a scaled-down bioreactor (e.g., smaller fermentation vessel, smaller supplies of starter material, nutrients and pH control agents), which makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

In one embodiment, the microbe growth facility is located on, or near, a site where the microbe-based products will be used (e.g., a citrus grove), for example, within 300 miles, 200 miles, or even within 100 miles. Advantageously, this allows for the compositions to be tailored for use at a specified location. The formula and potency of microbe-based compositions can be customized for specific local conditions at the time of application, such as, for example, which soil type, plant and/or crop is being treated; what season, climate and/or time of year it is when a composition is being applied; and what mode and/or rate of application is being utilized.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products to improve GHG management.

The cultivation time for the individual vessels may be, for example, from 1 to 7 days or longer. The cultivation product can be harvested in any of a number of different ways.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Novel Strain Meyerozyma Sp. MEC14XN

A Meyerozyma sp. microorganism was isolated as a laboratory contaminant in the inventors' microbial production facility (referred to herein as Meyerozyma sp. MEC2). Meyerozyma sp. MEC2 was determined to be closely related to Meyerozyma caribbica.

Meyerozyma sp. MEC2 was grown on Burk's plates, and over 14 iterations of clonal selection, a strain having enhanced nitrogen-fixation capabilities was obtained (referred to herein as Meyerozyma sp. MEC14XN).

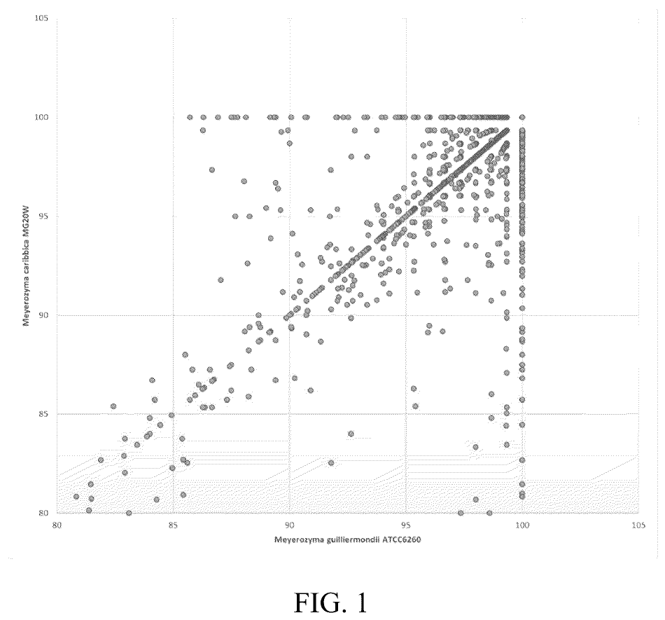

Meyerozyma sp. MEC14XN is a novel species having greater than 99% similarity to M. caribbica and M. guilliermondii. Whole-genome sequencing was performed, and results showed that a large portion of reads mapped to M. caribbica MG20W (relative abundance 53.55%) and M. guilliermondii ATCC6260 (relative abundance 46.07%), with a smaller portion of reads mapped to other Meyerozyma (relative abundance 1%). A set of 10,000 reads from the sample were randomly chosen to confirm results using BLAST runs against MG20W and ATCC6260. The overall average similarity to MG20W was 99.2%, the overall average similarity to ATCC6260 was 99.1%. FIG. 1.

### Example 2—Screening for Nitrogen Fixation

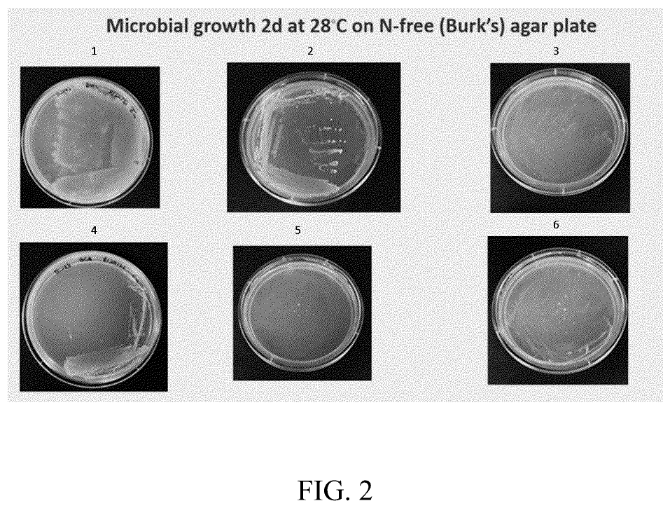

Meyerozyma sp. MEC14XN was compared to five other microorganisms that are known to grow in soil for nitrogen-fixation capabilities. As shown in FIG. 2, growth for 2 days at 28° C. on nitrogen-free (Burk's, Table 1) agar plates provided the following results for the 6 microbes based on visual observation.
1. B. subtilis NRRL B-68031: full biofilm coverage.
2. Meyerozyma sp. MEC14XN: good growth with isolated colonies.
3. Meyerozyma sp. MEC2: mild growth.
4. B. amyloliquefaciens NRRL B-67928: mild growth.
5. T. harzianum T-22: essentially no growth.
6. W. anomalus NRRL Y-68030: some growth.

TABLE 1
Burk's medium composition

| Component | g/L |
|---|---|
| CaSO4 | 0.13 |
| FeCl3 | 1.45 mg/L |
| K2HPO4 | 0.8 |
| KH2PO4 | 0.2 |
| MgSO4 | 0.2 |
| Na2MoO4 | 0.25 mg/L |
| Sucrose | 20 |

Add 15 g/L agar for solid medium.

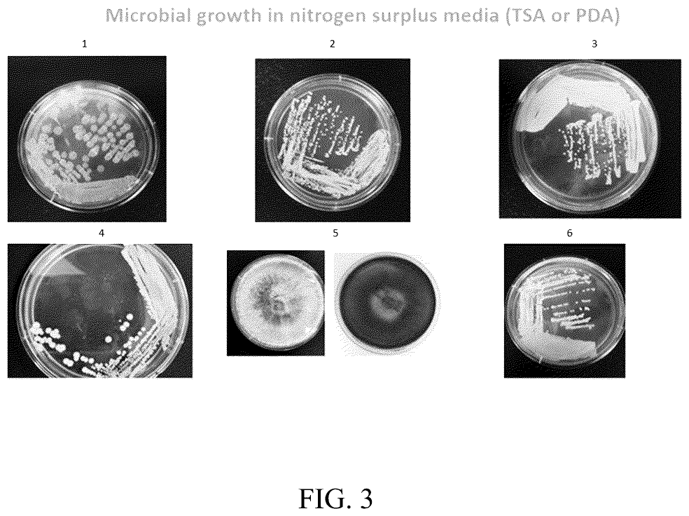

FIG. 3 shows growth of the 6 microorganisms grown on nitrogen-surplus media to show colony growth morphology under good conditions as a comparison:
1. B. subtilis NRRL B-68031: isolated colony growth (1 day; 37° C.).
2. Meyerozyma sp. MEC14XN: isolated colony growth (2 days; 28° C.).
3. Meyerozyma sp. MEC2: isolated colony growth (2 days; 28° C.).
4. B. amyloliquefaciens NRRL B-67928: isolated colony growth (1 day; 37° C.).
5. T. harzianum T-22: lawn growth (2 days, 8 days; 28° C.).
6. W. anomalus NRRL Y-68030: isolated colony growth (2 days; 28° C.).

### Example 3—Nitrogen Solubilization Comparison

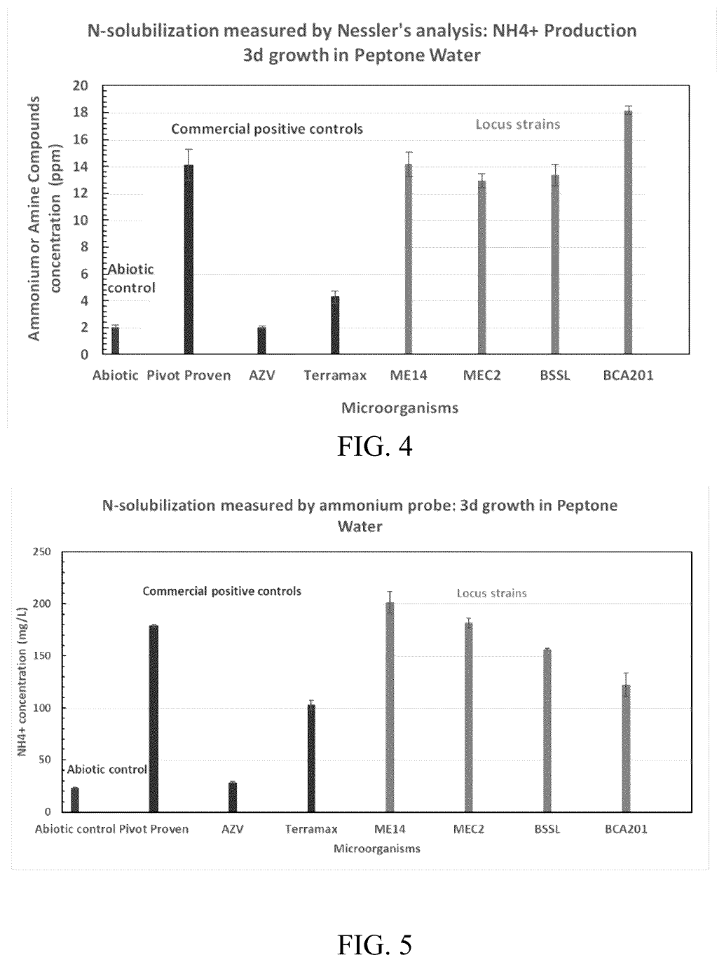

Nessler's analysis was used to quantify the total concentrations of ammonium and amine compounds grown for 3 days in peptone water for various microbes and commercial products. The results are reported in FIG. 4.

MECX14N (“ME14”) and B4 (“BSSL”) produced similar amounts of total ammonium and amine compounds to the leading competitive commercial product Pivot Proven®. B. amyloliquefaciens NRRL B-67928 (BCA201) produced the highest levels.

The results of the highest performing strains were verified using an ammonium ion probe. MECX14N produced a higher concentration of ammonium compared to other strains. FIG. 5.

### Example 4—N-Fixation and Solubilization Considering Fermentation Yield

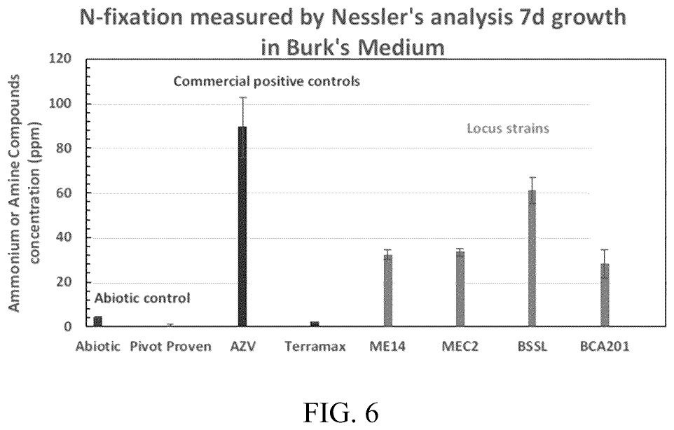

Microorganisms were grown in nitrogen-free Burk's Medium. Nessler's analysis was used to quantify the total concentration of ammonium and amine compounds produced. This was compared with average fermentation yield for each product, as shown in FIG. 6 and Table 3.

TABLE 3
N-fixation and solubilization yield considering fermentation yield.

| | | | | Pivot |
|---|---|---|---|---|
| Organism | MECX14N | B4 | Azotobacter | Proven |
| Average CFU/ml | 1.09 × 10\hat{ }10 | 3.79 × 10\hat{ }9 | 1.00 × 10\hat{ }8 | 4 × 10\hat{ }8 |
| Total N-fixation | 39.2 | 26.0 | 1 | 0 |
| yield considering | | | | |
| fermentation | | | | |
| yield (ratio) | | | | |
| Total N- | 30.7 | 8.3 | 0 | 1 |
| solubilization | | | | |
| yield considering | | | | |
| fermentation | | | | |
| yield (ratio) | | | | |

MEC14XN and B4 can deliver approximately 39X and 26X yields, respectively, for N-fixation as compared to Azotobacter, if considering fermentation yield.
MEC14XN and B4 can deliver approximately 31X and 8X yields respectively in N-solubilization as compared to Pivot Proven, if considering fermentation yield.

### REFERENCES

Brummell, M. E., and S. D. Siciliano. (2011). “Measurement of Carbon Dioxide, Methane, Nitrous Oxide, and Water Potential in Soil Ecosystems.” Methods in Enzymology. 496:115-137. Doi: 10.1016/B978-0-12-386489-5.00005-1. (“Brummell and Siciliano 2011”).
Grandy, A. S. and G. P. Robertson (2007). “Land-Use Intensity Effects on Soil Organic Carbon Accumulation Rates and Mechanisms.” Ecosystems 10:58-73. (“Grandy 2007”).
Kallenbach, C. M. et al. (2015). “Microbial physiology and necromass regulate agricultural soil carbon accumulation.” Soil Biol & Biochem 91:279-290. (“Kallenbach 2015”).
Kallenbach, C. M. et al. (2019). “Managing Agroecosystems for Soil Microbial Carbon Use Efficiency: Ecological Unknowns, Potential Outcomes, and a Path Forward.” Frontiers in Microbiol 10:1146. (“Kallenbach 2019”).
Panettieri, M. et al. (2013). “Moldboard plowing effects on soil aggregation and soil organic matter quality assessed by 13C CPMAS NMR and biochemical analyses.” Agric., Ecosys & Envt 177:48-57. (“Panettieri 2013”).
Possinger, A. R. et al. (2020). “Organo-organic and organo-mineral interfaces in soil at the nanometer scale.” Nature comm. 11:6103. (“Possinger 2020”).
Soil Survey Staff, USDA (2014). “Keys to Soil Taxonomy.” USDA Natural Resources Conservation Service. 12th Edition. (“USDA 2014”).
Trivedi, P. et al. (2015). “Soil aggregate size mediates the impacts of cropping regimes on soil carbon and microbial communities.” Soil Biol & Biochem 91:169-181. (“Trivedi 2015”).
Trivedi, P. et al. (2017). “Soil aggregation and associated microbial communities modify the impact of agricultural management on carbon content.” Envtl Microbiol 19 (8), 3070-3086. (“Trivedi 2017”).
Warncke, D. D., (2014). “Managing Muck Soils for Vegetable Crops.” Soil Fertility and Plant Nutrition, Michigan State University. (“Warncke 2014”). http://www.hort.cornell.edu/expo/proceedings/2014/Cover % 20Crops %20Tillage %20 and %20 Soils/Muck % 20Soils %20Warncke.pdf

1. A microbial fertilizer composition comprising a microorganism, a fermentation broth and/or solid-state substrate in which the microorganism was cultivated, and a growth by-product of the microorganism, wherein the microorganism is a nitrogen-fixing microorganism comprising a biological mechanism for converting atmospheric di-nitrogen into nitrate, ammonium salts, urea and/or ammonia.

2. The microbial fertilizer composition of claim 1, wherein the growth by-product is a nitrogen-fixing enzyme or an analog thereof.

3. The microbial fertilizer composition of claim 2, wherein the nitrogen-fixing enzyme is a nitrogenase or a flavodoxin.

4. The microbial fertilizer composition of claim 2, wherein the nitrogen-fixing enzyme is an enzyme complex comprising a reductase and a nitrogenase or an analog thereof.

5. The microbial fertilizer composition of claim 2, wherein the nitrogen-fixing enzyme is an enzyme complex comprising a reductase and a flavodoxin or an analog thereof.

6. The microbial fertilizer composition of claim 2, further comprising an electron donor.

7. The microbial fertilizer composition of claim 1, further comprising one or more biosurfactants.

8. The microbial fertilizer composition of claim 7, wherein the one or more biosurfactants are glycolipids, lipopeptides, flavolipids, phospholipids, fatty acid ester, lipoproteins, lipopolysaccharide-protein complexes, and/or polysaccharide-protein-fatty acid complexes.

9. The microbial fertilizer composition of claim 1, further comprising one or more organic and/or inorganic substrates selected from vegetable oil, glycerol, glucose, molasses, kelp extract, humic acid, fulvic acid, bone meal, blood meal, compost, corn gluten, potash, manure from a variety of animals including horses, cows, pigs, chickens, and/or sheep, nitrogen, phosphorus rock and/or potassium mica, and/or sources of calcium, magnesium, sulfur, boron, copper, ion, manganese, molybdenum and zinc.

10. The microbial fertilizer composition of claim 1, wherein the microbe is Ascoidea rubescens, Bacillus subtilis NRRL B-68031, Bacillus amyloliquefaciens NRRL B-67928, Brettanomyces bruxellensis, Brettanomyces naardenensis, Candida dubliniensis CD36, Candida intermedia, Candida maltosa Xu316, Candida viswanathii, Clavispora lusitaniae, Cyberlindnera jadinii NRRL Y-1542, Cyberlindnera fabianii, Debaryomyces hansenii CBS767, Geotrichum candidum, Kazachstania africana, Kazachstania saulgeensis, Kuraishia capsulata, Lachancea dasiensis CBS 10888, Lachancea lanzarotensis, Lachancea meyersii CBS 8951, Lachancea mirantina, Lachancea nothofagi CBS 11611, Lachancea thermotolerans CBS 6340, Lachancea quebecensis, Lodderomyces elongisporus NRRL YB-4239, Metschnikowia sp. JCM 33374, Metschnikowia bicuspidate, Meyerozyma sp. JA9, Meyerozyma caribbica subsp. Locus, Meyerozyma sp. MEC14XN, Meyerozyma guilliermondii ATCC 6260, Millerozyma farinosa CBS 7064, Ogataea parapolymorpha, Pachysolen tannophilus NRRL Y-2460, Saccharomyces eubayanus, Saccharomycodes ludwigii, Spathaspora passalidarum NRRL Y-27907, Sugiyamaella lignohabitans, Suhomyces tanzawaensis NRRL Y-17324, Tetrapisispora blattae CBS 6284, Tetrapisispora phaffii CBS 4417, Trichomonascus ciferrii, Wickerhamiella sorbophila, Wickerhamomyces anomalus NRRL Y-68030 or Wickerhamomyces anomalus NRRL Y-366-8.

11. The microbial fertilizer composition of claim 10, wherein the microorganism is Meyerozyma sp. MEC14XN or Meyerozyma guilliermondii.

12. The microbial fertilizer composition of claim 10, wherein the microorganism is Bacillus subtilis B4 NRRL B-68031.

13. The microbial fertilizer composition of claim 1, comprising 1×10⁶ to 1×10¹² CFU/ml of the yeast.

14. The microbial fertilizer composition of claim 1, further comprising a carrier selected from cornhusk, sugar industry waste, or crop residue.

15. The microbial fertilizer composition of claim 1, formulated as a liquid suspension, an emulsion, a freeze- or spray-dried powder, pellets, granules, gels, tablets, and/or capsules.

16. A method for enhancing plant health, growth and/or yields, wherein the method comprises applying a microbial fertilizer composition according to claim 1 to the plant and/or to soil in which the plant is growing or will be planted.

17. The method of claim 15, carried out at a temperature of 10° C. or less.

18. The method of claim 15, wherein the microbial fertilizer composition enhances nitrogen availability to plant roots compared to plants growing in untreated soils.

19. The method of claim 15, wherein the application of the microbial fertilizer reduces chemical fertilizer usage requirements.

20. The method of claim 15, wherein the application of the microbial fertilizer reduces nitrous oxide emissions from soil compared to untreated soils.
